(19) United States
(12) Patent Application Publication
(10) Pub. No.: US 2008/0207999 A1
(43) Pub. Date: Aug. 28, 2008

(54) Endoscopic Capsule

(76) Inventors: Klaus Abraham-Fuchs, Erlangen (DE); Rainer Kuth, Hochstadt (DE); Johannes Reinschke, Nurnberg (DE); Sebastian Schmidt, Weisendorf (DE)

Correspondence Address: SCHIFF HARDIN, LLP;PATENT DEPARTMENT, 6600 SEARS TOWER, CHICAGO, IL, 60606-6473, US

(21) Appl. No.: 11/994,150

(22) PCT Filed: Jul. 6, 2006
(86) PCT No.: PCT/EP2006/063978

§ 371 Date: Dec. 28, 2007

(30) Foreign Application Priority Data
Jul. 8, 2005 (DE) 10 2005 032 368.5

(51) Int. Cl. A61B 1/01

(52) U.S. Cl. 600/118

(57) ABSTRACT

An endoscopy capsule has a capsule housing containing at least one magnetic element that interacts with an extracorporeally applied magnetic field to magnetically navigate the endoscopy capsule within a body lumen of a patient. The capsule housing has a tube connected thereto that is composed of a flexible, non-rigid material, i.e., of insufficient rigidity to feed the capsule housing in the body lumen. The tube is provided with a feed path for providing any of a liquid agent, a gaseous agent, electrical power and data between the capsule housing an extracorporeal source.

### BACKGROUND OF THE INVENTION

1. Field of the Invention

The invention concerns an endoscopy capsule of the type having at least one magnetic element interacting with an external magnetic field for magnetic navigation of the endoscopy capsule.

2. Description of the Prior Art

For examination of the gastrointestinal tract a flexible catheter endoscope is typically used that is inserted orally or rectally and is advanced. A disadvantage of this technique is that the catheter is relatively stiff since the feed force must be passed along it. Such a forward shifting of the catheter tip means that regions further removed from the body orifice can be difficult to reach or, respectively, cannot be reached at all. Catheter endoscopy is relatively uncomfortable for the patient, it can lead to complications such as an organ perforation (when it is pressed too strongly against an organ wall), and the manual operation for the physician is also relatively elaborate and complicated.

As an alternative to this, the use of an endoscopy capsule is known that moves actively by means of an integrated magnetic element that interacts with a magnetic field (generated external to the patient) acting on the capsule, and with which the magnetic element is moved through the examination subject, meaning that the magnetic capsule navigation ensues by remote control, for example by actuation of a joystick or a mouse or the like. It is advantageous that an extensive automation of the medical procedure is possible. The automation capability has essentially two bases: the magnetic force effect ensues directly on the capsule; the perforation danger thereby drops drastically, and the control or the force no longer ensues directly manually, but rather indirectly via the control of the coil currents of the external magnetic system. The endoscopy capsule thus can be designed differently. It can be purely a video capsule that exhibits an image acquisition device with which images of the inside of the hollow organ can be acquired and transferred via radio to an external acquisition or control device. For example, a biopsy forceps or another mechanical instrument can be provided at the capsule, with the biopsy forceps or another mechanical instrument being externally controlled via radio in order to extract tissue samples or the like. In each case images and other measurement values or operations can be acquired or made at arbitrary locations in the gastrointestinal tract in this manner.

A disadvantage of catheter-free capsule endoscopy is that only limited resources for working or operating means or electrical energy can be carried by the capsule. A small battery that delivers only limited power is integrated therein for operating electrical loads such as an image acquisition device or the biopsy forceps or an electrical valve that connects a gas reservoir in the capsule with a balloon. If used, a gas quantity for inflation of the balloon (which, for example, serves for vessel widening or for setting a stent) as well as a possible fluid quantity (that, for example, is necessary for lavaging the intestinal wall or the like) as well as the quantity of a medicine that is to be applied on site, can be provided only in small quantities.

### SUMMARY OF THE INVENTION

An object of the present invention is to provide an endoscopy capsule that is no longer subject to the limitations described above that result from the limited carrying capability of working or operating means or from the limited power supply capacity.

This object is achieved in accordance with the invention by an endoscopy capsule of the aforementioned type equipped with a tube composed of a flexible and material, via which tube one or more fluid or gaseous operating or working agents can be supplied to the capsule, and/or in which at least one conductor serving for the signal and/or power line is directed to the capsule. As used herein, “non-rigid” means a rigidity that is insufficient to permit feed of the capsule to be done using the aforementioned tube.

The capsule is connected with external supply or feed devices via the thin, highly flexible supply tube, such that a continuous feed of necessary operating or working agents or a power feed is possible. The excellent navigability of the capsule with all of its advantages is retained; the capsule merely pulls the thin non-feed rigid tube behind it, which does not affect the mobility via the external magnetic field navigation device since the thin, highly flexible tube slides along the organ walls without further measures and can be pulled along through corresponding intestinal coils or the like without further measures. The tube, which preferably is formed of an inelastic (i.e. bendable but not expandable) material such as polypropylene or polytetrafluorethylene so that it does not elastically expand (for example given feed of a gaseous operating or working means) can be executed with very thin walls and very small in terms of diameter; a feed or, respectively, supply or, respectively, communication is nevertheless possible without further measures. The wall thickness of the tube can be between 0.1-0.5 mm (preferably 0.2 mm) while the outer diameter of the tube can be between 2-6 mm (in particular 3-4 mm). The own weight of the tube is extremely low and lies in the range of a few grams, even when the tube is executed very long. Lengths of more than 2 m are realizable without further measures; a length of up to 8 m is also conceivable, such that the tube can be drawn through the length of the entire gastrointestinal tract.

Two or more separate channels sealed off from one another are advantageously fashioned in the tube (which should have a sufficient tensile strength so that it, together with the endoscopy capsule, can be pulled out from the gastrointestinal tract undamaged as needed), via which separate channels the various operating or working means can even be supplied simultaneously if needed. The corresponding channels are naturally directed at the capsule to the corresponding function devices of the capsule that should be supplied with the respective operating or working means, whereby the channels or continuation lines can be opened and closed as needed via corresponding electrical valves controllable via a capsule-side control device. For example, it is possible to feed a gas via a first channel, by means of which gas a balloon arranged at the capsule is inflated. By means of this balloon the capsule size (thus the capsule diameter) can be adapted to the size of the surrounding hollow organ for a sliding-contacting [sic] movement of the capsule along the organ wall, is fed, or via which a stent or a tamponade or the like can be placed, for example. Via the second channel a ravaging fluid that exits at a capsule-side exit opening (in order, for example, to clean the intestinal wall or the viewing window of an optical sensor in the capsule) can be fed, for example.

The at least one (but typically more) electrical conductor is appropriately set in the tube wall, but can also be directed on the tube wall. In the case of a power supply, only very slight currents are to be conducted via these conductors. The communication between the external operating or control device and the capsule-side control device can also ensue via these same conductors, i.e. the image and other measurement data that are acquired at the capsule can be transferred to the external operating or control device, or control commands can be provided from the outside to capsule-internal function devices.

As stated, at least one outlet opening for a supplied working or operating agent can be provided at the capsule, this outlet opening being advantageously positioned adjacent to an image acquisition device integrated into the capsule. For example, for an improved image acquisition a cleaning fluid can thus be supplied from outside and can be emitted via the outlet opening directly at the location of the Office Action. A number of such outlet openings can naturally also be provided. The tube-side channel opening at the capsule would then be coupled with the respective outlet openings via a corresponding connection channel system. Here as well a closing and opening of the respective channels or outlet openings via electrically controlled valves is naturally appropriate. The cleaning openings can also be combined with other sensors or probes on the capsule surface, for example a conductivity sensor or a bipolar probe for thermal coagulation.

In the event a working or operating agent cannot be supplied via the very thin tube with the sufficient pressure that would be required for a sufficient washing of the intestinal wall or for a sufficiently strong inflation of a balloon or the like, in an embodiment of the invention a reservoir is provided for the supplied working or operating agent in the capsule, from which reservoir the working or operating agent can be removed via a pump or the like for output to a function device of the capsule or into the capsule environment. The reservoir can thus be continuously filled from the outside, while via the pump sufficient pressure can be developed so that the working or operating agent can perform its function.

In addition to the extraction of tissue samples via a biopsy device, it is also sometimes appropriate to acquire liquid or gas samples from the examination location, for example. For this purpose, a suction device for suction of fluid or gas from the capsule environment via a capsule-side inlet opening and for feeding the fluid or gas into the tube (possibly the reservoir) is appropriately provided. The corresponding inlet opening (which, as described, can be opened and closed via an electrically controllable valve) thus enables the immediate acquisition of local fluid or local gas that can then be transported out with the capsule. The same acquisition can naturally ensue via an outlet opening provided anyway, which outlet opening is, for example, coupled with the pump already described, this pump can then be operated in reverse as a suction device.

As described above, the opening and closing of the outlet and inlet openings or of connection lines leading to function devices ensues via corresponding valves that are electrically controllable via a control device integrated into the capsule. Insofar as no electrical communication line to an external operating device is provided, this control device can also communicate wirelessly via radio with the external operating or control device (alternatively via the tube-side signal lines, naturally). The control device (a small microprocessor) controls all electrically controllable or operating functions or operating elements that are integrated into the capsule.

Because the capsule sometimes rotates around its own axis during the magnetic navigation, it is appropriate when a coupling element at which the tube is attached is arranged at or in the capsule, and said coupling element enables a rotation of the capsule relative to the tube. The capsule can thus rotate freely relative to the tube, which does not have to track the capsule rotation movement; it thus does not twist. The coupling element is designed such that naturally the corresponding conductor connections from the tube to the capsule are also not interrupted upon rotation. The coupling element itself does not necessarily have to be arranged at the point at which the tube discharges into the capsule; rather, the coupling can also be provided at an arbitrary point along the tube, preferably close to the capsule, naturally.

Furthermore, it is sometimes appropriate to be able to decouple the tube from the capsule as needed, which can possibly ensue via the coupling element. For example, this can ensue via an electrical signal given by the capsule-side control device, which electrical signal specifically opens a mounting at the coupling element or at the connection of the tube with the capsule, or, for example, by defined mechanical pull on the tube, such that a connection mechanism between tube and capsule is hereby opened in a defined manner. The tube can then be drawn out while the capsule (which, for example, requires no further supply with working or operating means or the like) can be further directed through the intestine or the like via external control. Alternatively, the tube can also remain in the body in order to be used as a feeding or drainage tube while the endoscopy capsule is no longer needed. In this case the capsule can then be magnetically navigated further and secured. Here the accessibility of the entire intestine via the navigable capsule proves to be particularly advantageous, such that in the case of ileus (for example) a discharge sample can be placed very far aboral (for example in the jejunum or ileum) or a feeding tube can be introduced through the colon into the small intestine given a failing continuity of the oral sections of the gastrointestinal tract.

Given use of tubes that are shorter than the entire length of the gastrointestinal tract, given oral examinations (gastroscopies) the decoupling capability offers the possibility to remove the tube without pain via mouth or nose after decoupling while the capsule is navigated further or moves via natural peristalsis and is secured anally. It is also sometimes possible to leave the capsule inside the gastrointestinal tract (possibly locally fixed) for further gastroenterological examination or treatment, however to already remove the tube because no further working or operating means or, respectively, energy supply is required.

In a further embodiment of the invention the magnetic element is arranged in a housing section that can be decoupled from the remaining capsule housing as needed. This enables the magnetic element to be retrieved via the tube after the positioning of the endoscopy capsule in a target region, meaning that the decoupling-capable housing section is connected with the tube and can be drawn out with this. This enables the patient to be examined in a magnetic resonance system after the positioning of the endoscopy capsule since, given corresponding design, after removal of the magnetic element the endoscopy capsule no longer contains components that would react to the magnetic fields predominating during the magnetic resonance examination. It is also conceivable to direct a further magnetic endoscopy capsule via magnetic control to the same location, whereby the already-positioned capsule no longer interacts with the navigation field, i.e. is no longer displaced into movement with the navigation field. The detaching of the housing section from the remaining housing can ensue in manner described above as with the tube decoupling.

Furthermore, an insertion element (for example a tube or the like) to be inserted into a body orifice of an examination subject (for example the rectum) can be associated with the endoscopy capsule, via which insertion element the capsule can be inserted into the examination subject, and the insertion element exhibits an arresting and/or advancement and retraction device for the tube. By means of the arrest the capsule can “dangle” on the tube in an intestinal section directed downwards; a magnetic levitation is not necessary. Particularly given the retrograde capsule movement, the pulling device in the insertion element can support the magnetic capsule navigation when both “movement types” (magnetic force on the capsule and drawing on the tube) are exerted with adjustment to one another.

An easier capsule navigation is thus possible via the insertion element. The arresting and/or advancement and retraction device can be manually or mechanically actuated, however can also be controlled automatically and electrically.

The insertion element itself can be executed gastight in order to enable a filling of the colon with gas to enlarge the same.

### BRIEF DESCRIPTION OF THE DRAWINGS

FIG. 1 is a schematic block diagram of an endoscopy capsule in accordance with the present invention.

FIG. 2 is a section through the flexible, non-rigid feed tube of the endoscopy capsule shown in FIG. 1.

### DESCRIPTION OF THE PREFERRED EMBODIMENTS

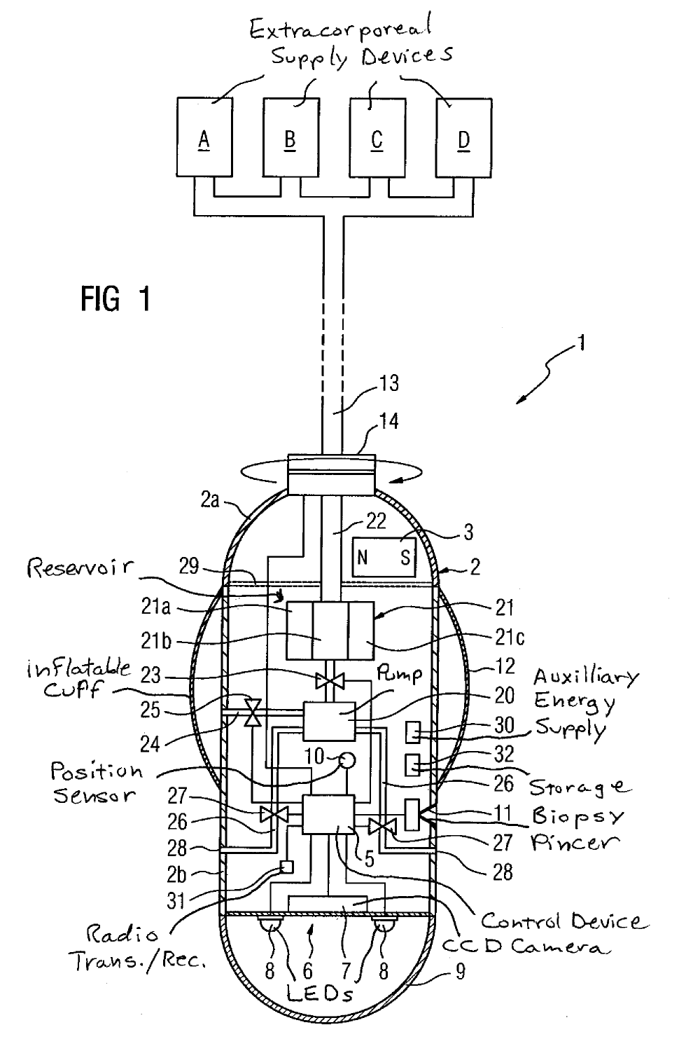

FIG. 1 shows an inventive endoscopy capsule 1 with a capsule housing 2 in which is integrated a magnetic element 3, which can be a permanent magnet, a weakly magnetic element that can be magnetized in a magnetic field, or an electronic coil. This magnetic element 3 interacts with navigation magnetic fields that are generated via an external navigation device (not shown) so that the endoscopy capsule 1 accommodated in the patient body can be actively directed and moved via external control.

A control device 5 in the form of a microcontroller is integrated into the oblong, cylindrical capsule exhibiting a diameter of, for example, 10 mm, which control device 5 takes over all control tasks concerning the function devices of the capsule (which are subsequently described in detail). An image acquisition device 6 is also provided, comprising a camera (for example a CCD camera 7 with which two illumination devices are associated in the form of two LEDs 8). Via the image acquisition device 6 (that is arranged behind a transparent capsule window covering 9) it is possible to acquire images of the examination volume that is illuminated via the LEDs 8. The image signals are passed to the control device 5 which transfers these outward via a conductor connection to an external control or operating device (as is described further in the following).

The detection of the position of the capsule inside the examination subject ensues in connection with a position sensor 10 provided at the capsule, which position sensor 10 interacts with a magnetic position detection system (not shown in detail). Also provided in the shown example is a function device in the form of a biopsy pincer 11 that can be controlled via the control device 5 in order to extract tissue samples. Finally a balloon or cuff 12 that can be reversibly inflated (which is discussed further in the following) is arranged at the capsule housing 2. The outside of the capsule can be adapted or altered via this cuff 12 in order to adapt to changing diameters of the hollow organ examined or to be examined.

The endoscopy capsule 1 also has or is also connected with a highly flexible, non-feed rigid tube 13 via a coupling element 14. This tube comprises polypropylene (PP) or polytetrafluorethylene (PTFE), thus an inelastic material that does not expand given internal pressurization, and is also extremely thin in diameter with regard to the wall thickness. The latter is preferably approximately 0.2 mm; the diameter is preferably 3-4 mm. This tube 13, which can be two or more meters long, is inserted into the patient together with the endoscopy capsule. The endoscopy capsule, as it is actively moved forward, draws the tube after it. The tube itself is extremely smooth on the outside, thus in practice slides along the organ wall without resistance and follows any curve without further measures because as executed it is extremely thin-walled and highly flexible.

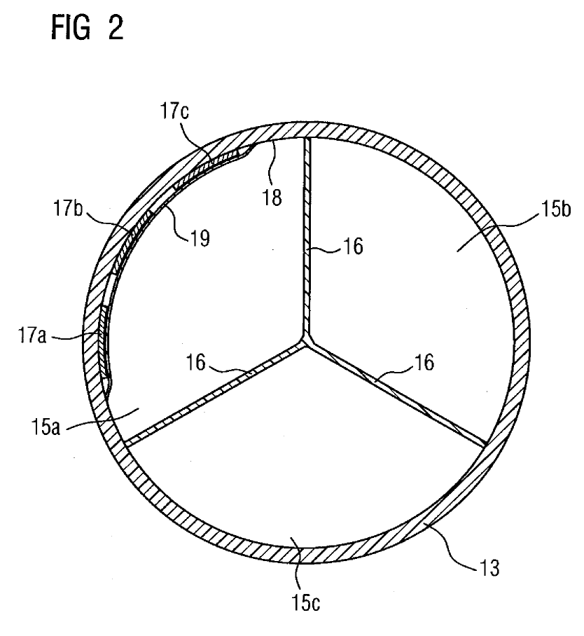

Inside the tube (see FIG. 2) three different lumens or channels 15a, 15b and 15c are demarcated from one another via corresponding dividing walls 16. Via these channels 15a-15c it is possible to direct different working or operating means from the outside to the endoscopy capsule 1 which requires these in some form, thus requires these for internal operation or would like to emit them externally into the examination organ. For example, a CO₂ gas can hereby be fed as a washing gas that is emitted at the capsule into the intestine via an outlet opening. Water can also be supplied as a washing solution, or a medicinal substance that is emitted externally. Furthermore, the gas needed to inflate the balloon 12 can hereby be supplied. For this one the channel or channels are coupled with corresponding lines inside the capsule that lead to the function devices or outlets where the working or operating means are required (which is discussed further).

Furthermore, a number of electrical conductors 17a, 17b, 17c are shown that, in the shown example, are directly attached to the inner wall 18 of the tube 13 as thin-film conductors and that, in the shown example, are sealed off from the channel 15a with a thin membrane 19. Via these electrical conductors it is possible on the one hand to ensure the power supply of the electrical loads inside the capsule. For example, the conductor 17a serves for this, which conductor 17a is correspondingly looped further inside the capsule and is connected with the corresponding loads such as the control device 5, the image acquisition device 6 with its components or the biopsy pincer 11, but also a pump integrated into the capsule (which is subsequently discussed further). For example, the conductor 17b serves for bidirectional signal or data transfer. For example, the communication between an external control or operating device and the control device 5 can thus ensue via the conductor 17b. The conductor 17c is, for example, a common neutral conductor for the conductors 17a and 17b. Image signals acquired via the image acquisition device 6 can be transferred from the control device 5 (for example via the conductor 17b) to the external control or operating device that processes and prepares the image signals and outputs them onto an associated monitor.

The inventive endoscopy capsule 1 is thus clearly not autarkic, meaning it does not carry the necessary working or operating means with it; rather, in the shown example it is supplied from the outside with all required working or operating means including the necessary electrical current. This supply occurs via the highly flexible, extremely thin tube (serving exclusively as a connection element) that is drawn behind the capsule and that otherwise has no function whatsoever with regard to the mechanical capsule movement. Rather, the capsule movement ensues exclusively via the magnetic navigation.

As stated, a pump 20 is integrated inside the capsule, upstream from which pump 20 is a reservoir 21 that is coupled via a line connection section 22 with the tube 13 that leads to the coupling element 14. In the shown example the reservoir 21 exhibits three separate chambers 21a, 21b and 21c into which a channel 15a, 15b or 15c respectively leads. The supplied working or operating means (thus for example a flushing gas or a cleaning fluid or the like) can be cached [buffered] in said reservoir 21 and be removed as needed via the pump 20, upstream from which is a multi-path valve 23 that can be correspondingly switched via the control device 5. The pump 20 can generate the higher (compared with the feed pressure possible due to the extremely low channel diameter) pressure sometimes required, which is required for example in order to enable a sufficient washing or to inflate the cuff 12. At this point it is noted that the reservoir 21 can naturally also be omitted if, for example, the feed should be possible with sufficiently high pressure when, for example, only one channel is provided at the tube and different working or operating agents are supplied via this, for example sequentially.

In the shown example diverse lines exit from the pump 20 to different function devices. A first line 24 with integrated valve 25 that can be controlled via the control device 5 opens below the balloon 12. If this should be inflated, the pump 20 pumps the corresponding gas supplied via the tube 13 (possibly after preceding extraction from the reservoir 21) into the balloon and inflates this.

Two further lines 26 with associated valves 27 switchable via the control device 5 open at the capsule housing 2 in the openings 28 just before the image acquisition device 6. They serve for the deployment of washing gas or washing fluid that is conveyed via the pump 20 with relatively high pressure. Given reverse operation of the pump it is also possible when this thus acts as a suction pump to draw liquid or gas from the capsule environment (thus from the hollow organ) into the capsule and, for example, to store it in the reservoir 21 from where it can be extracted and examined when the capsule is secured.

At this point it is noted that the pump 20, like the reservoir 21, is naturally only optional. If, as stated, a feed of the working or operating means with sufficient pressure should be possible, these elements are not required; rather, the required CO₂ gas for inflation of the balloon can be supplied directed by a corresponding external feed controller and be conducted into the balloon, or, respectively, the flushing gas can then be directed directly to the openings 28 (that, as stated, can serve as outlet or inlet openings).

The coupling element 14 is fashioned such that a rotation of the capsule 1 around its longitudinal axis relative to the stationary tube is possible, meaning that it is a swivel coupling (as is shown by the arrow). This enables the tube 13 to not have to follow possible capsule rotations around the capsule longitudinal axis (not drawn). This embodiment is particularly suitable when the tube 13 has only one channel. Otherwise it must be ensured that, in spite of capsule rotation, the connection of the tube-side channels with the corresponding connections inside the capsule is maintained. The electrical connection can be realized by slip ring connections or the like in the coupling element 14.

In order to enable the detachability of the tube 13 from the endoscopy capsule as needed, the coupling element 14 can be controlled via the control device 5 so that an opening mechanism (not shown in detail) integrated into the coupling element is activated and the tube 13 is decoupled. This can hereby be a simply fashioned, electrically controllable mechanism. This enables the tube to be detached from the capsule as needed, the tube to be withdrawn and the capsule to be directed further etc. Additionally or alternatively, it is also conceivable to separate the upper capsule housing 2a which directly connects to the coupling element 14 and which is connected with the lower capsule housing 2b via a sealed dividing wall 29 (shown here only dashed). Exclusively the magnetic element 3 is arranged in the upper capsule housing 2a. Thus upper capsule part together with the magnetic element 3 can thus be removed as needed so that only the lower capsule part 2b remains in the body. The remainder can be withdrawn with the tube 13. This offers the possibility to leave the capsule in the body during a magnetic resonance examination.

In order to generally maintain the operation of the capsule even when the tube 13 is decoupled, it is moreover conceivable to integrate an auxiliary energy supply 30 into the capsule so that it is ensured that, for example, the image acquisition device can also still operate after the decoupling. The radio transmitter/receiver 31, which wirelessly transmits the image signals outside to the operating or control device and/or receives control signals for opening or closing of the valves 23, 25, 27, then serves, for example, to transfer the acquired images and receive external control signals. It is also possible to optionally provide one or more storages 32 for gas or liquid or the like from which a certain albeit small quantity can be removed and employed in case of need given a decoupled tube. This in particular lends itself when the optional reservoir 21 is not provided. The storage or storages 32 are naturally connected with the remaining line system via corresponding lines (not shown in detail).

As FIG. 1 also shows, the tube 13 is connected at its external end with a plurality of external supply or operating or control devices. In the shown example, for example, the supply devices A, B and C are connected with the channels 15a, 15b and 15c via which a corresponding working or operating means can be supplied in a gaseous or liquid form. D exemplarily identifies the external control or operating device via which the entire capsule operation can be controlled (i.e. the electrical current feed and the data exchange can ensue) and that is connected with the capsule via the conductors 17a, b, c.

Although modifications and changes may be suggested by those skilled in the art, it is the intention of the inventors to embody within the patent warranted heron all changes and modifications as reasonably and properly come within the scope of their contribution to the art.

We claim as our invention:

1.-15. (canceled)

16. An endoscopy capsule comprising:
a capsule housing configured for in vivo movement through an anatomical lumen of a patient;
at least one magnetic element in said capsule housing that interacts with an externally applied magnetic field to participate in magnetic navigation of the capsule housing through said anatomical lumen;
a tube connected to said capsule housing said tube being comprised of a flexible, non-rigid material;
a component in said capsule housing that participates in an interaction with the patient while the capsule housing is in said anatomical lumen, said component requiring communication with an extracorporeal component in order to participate in said interaction; and
said tube being configured to provide communication to said extracorporeal component and comprising a feed path between said extracorporeal component and said component in said capsule housing selected from the group consisting of a feed path for a fluid agent, a feed path for a gaseous agent, a feed path for electrical supply current, and a feed path for data signals.

17. An endoscopy capsule as claimed in claim 16 wherein said tube is comprised of an inelastic material selected from the group consisting of polypropylene and polytetrafluoroethylene.

18. An endoscopy capsule as claimed in claim 16 wherein said tube has a tube wall with a wall thickness in a range between 0.1 and 0.5 mm.

19. An endoscopy capsule as claimed in claim 18 wherein said tube wall has a thickness of 0.2 mm.

20. An endoscopy capsule as claimed in claim 16 wherein said tube has an external diameter in a range between 2 and 6 mm.

21. An endoscopy capsule as claimed in claim 20 wherein said tube has an external diameter in a range between 3 and 4 mm.

22. An endoscopy capsule as claimed in claim 16 wherein said tube comprises at least two separate channels that are sealed and separated from each other in said tube, respectively forming two different ones of said feed paths in said group.

23. An endoscopy capsule as claimed in claim 16 wherein said tube comprises a tube wall, and wherein said feed path comprises an electrical conductor attached to an interior of, or embedded in, said tube wall.

24. An endoscopy capsule as claimed in claim 16 wherein said feed path is a feed path for said gaseous agent or said liquid agent, and wherein said housing capsule comprises a channel therein terminating in a discharge port at an exterior of said capsule housing, said channel placing said feed tube in communication with said discharge opening to allow said gaseous fluid or said liquid fluid to be discharged through said discharge opening.

25. An endoscopy capsule as claimed in claim 24 comprising a reservoir and a pump connected between said feed tube and said discharge opening, said reservoir allowing accumulation of a quantity of said gaseous agent or liquid agent therein, and said pump operating to pump at least a portion of said quantity from said reservoir to said discharge opening.

26. An endoscopy capsule as claimed in claim 25 wherein said pump is operable in a first direction to pump said quantity from said reservoir to said discharge opening, and is operable in a second, opposite direction to draw said quantity from said feed line into said reservoir.

27. An endoscopy capsule as claimed in claim 25 wherein said pump has an input line communicating said pump with said reservoir and an output line communicating said pump with said discharge opening, and comprising at least one electrically controllable valve selected from the group consisting of a valve located in said input line and a valve located in said output line, said at least one electrically controllable valve being operated to control passage of said quantity from said reservoir to said discharge opening.

28. An endoscopy capsule as claimed in claim 16 wherein said component is a component requiring said liquid agent or said gaseous agent in order to participate in said inner action, and comprising a pump connected between said component and said feed line to pump said fluid agent or said gaseous agent from said feed line to said component.

29. An endoscopy capsule as claimed in claim 28 comprising a reservoir and a pump connected between said feed tube and said discharge opening, said reservoir allowing accumulation of a quantity of said gaseous agent or liquid agent therein, and said pump operating to pump at least a portion of said quantity from said reservoir to said discharge opening.

30. An endoscopy capsule as claimed in claim 16 wherein said feed path comprises an electrical conductor having a current-carrying capacity, and comprising a power source in said capsule housing connected to said electrical conductor, said power source providing an output power that is larger than a power associated with the current-carrying capacity of the electrical conductor, and said power source accumulating said output power from the power associated with the current-carrying capacity of the electrical conductor.

31. An endoscopy capsule as claimed in claim 16 comprising a coupling element that couples said tube to said capsule housing and allows rotation of said capsule housing relative to said tube.

32. An endoscopy capsule as claimed in claim 16 comprising a decoupleable attachment element connecting said tube to said capsule housing and allowing decoupling of said tube from said capsule housing.

33. An endoscopy capsule as claimed in claim 16 wherein said capsule housing comprises a main housing section and a detachable housing section that is detachable from said main housing section, and wherein said component is contained in said detachable housing section.

34. An endoscopy capsule as claimed in claim 16 comprising an insertion element attached to said capsule housing, said insertion element mechanically controlling movement of said capsule housing relative to said anatomical lumen to perform movement functions selected from the group consisting of advancement of said capsule housing into said anatomical lumen, retraction of said capsule housing from said anatomical lumen, and a resting movement of said capsule housing in said anatomical lumen.
